(19) United States
(12) Patent Application Publication
(10) Pub. No.: US 2016/0367688 A1
(43) Pub. Date: Dec. 22, 2016

(54) RAMAN-ACTIVE POLYMER PARTICLES AND METHODS FOR SYNTHESIZING THEREOF

(71) Applicant: THE TRUSTEES OF COLUMBIA UNIVERSITY IN THE CITY OF NEW YORK, New York, NY (US)

(72) Inventors: LUIS MIGUEL CAMPOS, Brooklyn, NY (US); Wei Min, Edgewater, NJ (US); Tristan Lambert, New York, NY (US); Fanghao Hu, New York, NY (US); Spencer Daniel Brucks, Brooklyn, NY (US)

(21) Appl. No.: 15/183,489

(22) Filed: Jun. 15, 2016

Related U.S. Application Data
(60) Provisional application No. 62/175,895, filed on Jun. 15, 2015.

(51) Int. Cl. A61K 47/48

(52) U.S. Cl. CPC A61K 47/48176; A61K 47/489; A61K 47/48569; A61K 47/48061

(57) ABSTRACT

An exemplary Raman-active nanoparticle can be provided, which can include, for example, a hydrophilic cyclopropenium cation, and one or more copolymers derived from a hydrophobic organic polymerizable molecule, where the hydrophobic organic polymerizable molecule can be a Raman-active molecule, and where the Ramain-active nanoparticle can be free of heavy metals. In some exemplary embodiments of the present disclosure, the hydrophobic organic polymerizable molecule can be a styrenic derivative. The styrenic derivative can be an alkyne, a nitrile, or a deuterated styrene. The hydrophobic organic polymerizable molecule can be a methacrylate derivative.

### CROSS-REFERENCE TO RELATED APPLICATION(S)

This application relates to and claims priority from U.S. Patent Application No. 62/175,895, filed on Jun. 15, 2015, the entire disclosure of which is incorporated herein by reference. Additionally, this application relates to International Application No. PCT/US15/44331, the entire disclosure of which is also incorporated herein by reference.

### STATEMENT REGARDING FEDERALLY SPONSORED RESEARCH

This invention was made with government support under Graduate Research Fellowship No. 11-4415 and Grant No. DMR-1351293, awarded by the National Science Foundation. The government has certain rights in the invention.

### FIELD OF THE DISCLOSURE

The present disclosure relates generally imaging of Raman-active polymer particles, and more specifically, to exemplary embodiments of Raman-active polymer particles and methods for synthesizing thereof.

### BACKGROUND INFORMATION

Cationic polyelectrolytes hold transformative potential in applications as diverse as energy storage and therapeutics. (See, e.g., Reference 1). Over the last decade, extensive research efforts have focused on exploiting these systems as ion-conducting membranes (see e.g., Reference 2) and gene delivery vectors. (See e.g., Reference 3). However up to this point, cationic materials have been primarily restricted to modalities bearing formal charge on heteroatoms. (See e.g., Reference 4). Leading monomeric functionalities such as ammonium (see e.g., Reference 5), imidazolium (see e.g., Reference 6), and phosphonium (see e.g., References 7 and 8), can be valuable but lack broad modularity; in such materials, coulombic interactions can be primarily tuned through variation of the counterion. Readily accessible materials possessing inherent compositional modularity can be of particular import for the advancement of the field. (See, e.g., References 9-11).

Thus, it is beneficial to provide exemplary Raman-active polymer particles which can overcome at least some of the deficiencies described herein above.

### SUMMARY OF EXEMPLARY EMBODIMENTS

These and other objects, features and advantages of the exemplary embodiments of the present disclosure will become apparent upon reading the following detailed description of the exemplary embodiments of the present disclosure.

An exemplary embodiment of the present disclosure can include, a Raman-active nanoparticle which can include, for example, a hydrophilic cyclopropenium cation, and one or more copolymers derived from a hydrophobic organic polymerizable molecule, where the hydrophobic organic polymerizable molecule can be a Raman-active molecule, and where the Ramain-active nanoparticle can be free of heavy metals. In some exemplary embodiments of the present disclosure, the hydrophobic organic polymerizable molecule can be a styrenic derivative. The styrenic derivative can be an alkyne, a nitrile, or a deuterated styrene. The hydrophobic organic polymerizable molecule can be or can include a methacrylate derivative.

In certain exemplary embodiments of the present disclosure, the Raman-active nanoparticle can further include a block copolymer electrolyte. The block copolymer electrolyter can further include a styrenic derivative, which can be alkyne, nitrile or deuterated styrene. The functionalized chemical linker can be be an N-hydroxysuccinimide ester. The Raman-active nanoparticle can be conjugated to an antibody, or a drug and the drug can be for breast cancer. The Raman-active nanoparticle can further include a nucleic acid conjugated by electrostatic interactions, where the nucleic acid can be RNA or DNA.

According to a further exemplary embodiment of the present disclosure, a method of treating and visualizing a tumor in a patient in need thereof can be provided, which can include, for example, synthesizing a Raman-active nanoparticle free of heavy metals, where the Raman-active nanoparticle can include a hydrophilic cyclopropenium cation and one or more copolymers formed from a hydrophobic organic polymerizable molecule, and where the hydrophobic organic polymerizable molecule can be styrenic derivatives or methacrylate derivatives. The Raman-active nanoparticle can be functionalized with a chemical linker, conjugated to an antibody or drug suitable for treatment of the tumor, administered to the patient and visualized at the location of the tumor by stimulated Raman scattering.

In some exemplary embodiments of the present disclosure, the antibody targets cell surface proteins specifically expressed by the tumor. The drug can be suitable for treatment of breast cancer, prostate cancer, colon cancer, liver cancer, skin cancer or lung cancer. The chemical linker can include one of N-hydroxysuccinimide ester, disulfide linkers, or non-cleavable methyl ester linkers. The styrenic derivatives can include one of analkyne, a nitrile or a deuterated styrene.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1A is an exemplary diagram of an exemplary structure of the cyclopropenium (CP) ion with modular handles according to an exemplary embodiment of the present disclosure;

FIG. 1B is an exemplary diagram of an exemplary molecule of a CP-based polyelectrolyte family according to an exemplary embodiment of the present disclosure;

FIG. 2A is an exemplary diagram of CP monomers according to an exemplary embodiment of the present disclosure;

FIG. 2B is an exemplary diagram and a chart showing how the exemplary molecule can stabilize an oil in water emulsion to form nanoparticles of controllable size according to an exemplary embodiment of the present disclosure;

FIG. 2C is an exemplary diagram of CP-based block copolymer electrolytes (BCPEs) according to an exemplary embodiment of the present disclosure;

FIG. 3 is an exemplary image showing Raman spectroscopy of CP nanoparticles (305) and lipids (310) according to an exemplary embodiment of the present disclosure; and

FIG. 4A is an exemplary energy diagram according to an exemplary embodiment of the present disclosure;

FIG. 4B is an exemplary graph illustrating stimulated Raman spectroscopy with a coincident Pump and Stokes beam according to an exemplary embodiment of the present disclosure;

FIG. 4C is a set of exemplary images of vibrations corresponding to methylene (CH₂) and methyl (CH₃) vibrations according to an exemplary embodiment of the present disclosure;

FIG. 5 is an exemplary diagram illustrating various exemplary monomer structures that can be used to synthesize the Raman-active monomers according to an exemplary embodiment of the present disclosure;

FIG. 6 is an exemplary diagram illustrating unmodified nanoparticle synthesis according to an exemplary embodiment of the present disclosure;

FIG. 7 is an exemplary diagram illustrating the synthesis of an exemplary nanoparticle with an alkyne tag according to an exemplary embodiment of the present disclosure;

FIG. 8 is an exemplary diagram illustrating the synthesis of a nanoparticle with a nitrile tag according to an exemplary embodiment of the present disclosure;

FIG. 9 is an exemplary diagram illustrating the synthesis of a nanoparticle with a carbon-deuterium tag according to an exemplary embodiment of the present disclosure;

FIG. 10 is an exemplary graph illustrating the Raman spectra for all synthesized nanoparticles according to an exemplary embodiment of the present disclosure;

FIG. 11 is an exemplary diagram illustrating a synthetic procedure for designing a block copolymer that has an N-hydroxysuccinimide ester tag by starting with a N-hydroxysuccinimide atom-transfer radical polymerization initiator and polymerizing two monomers in sequence according to an exemplary embodiment of the present disclosure;

FIG. 12 is an exemplary diagram illustrating the synthetic scheme procedure from FIG. 11 with an additional step of synthesizing the nanoparticle that has the N-hydroxysuccinimide ester tags on the periphery according to an exemplary embodiment of the present disclosure;

FIG. 13 is an exemplary flow diagram of an exemplary method for treating and visualizing a tumor in a patient in need thereof according to an exemplary embodiment of the present disclosure; and

FIG. 14 is an illustration of an exemplary block diagram of an exemplary system in accordance with certain exemplary embodiments of the present disclosure.

Throughout the drawings, the same reference numerals and characters, unless otherwise stated, can be used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures.

### DETAILED DESCRIPTION OF EXEMPLARY EMBODIMENTS

The exemplary method can be utilized to synthesize and image polymer particles with Raman-active tags. For example, the particles can be synthesized by an emulsion polymerization strategy, using, for example, cyclopropenium-containing surfactants and Raman-active comonomers (e.g. by the synthesis process described in International Patent Application PCT/US2015/044331, which is incorporated herein by reference in its entirety). The Raman-active comonomer can be covalently incorporated into the nanoparticle core with minimum influence on particle behavior. Carbon-deuterium bonds, alkynes or nitriles, among other units, such as styrenic derivatives, methacrylate derivatives, or any other organic polymerizable molecules, can be used as the functional Raman tag. The Raman-active nanoparticles of the present invention preferably do not include heavy metals.

Each Raman tag has an inherent molecular vibration that may not be present in the cellular environment. The phenyl ring present in nanoparticles synthesized without a Raman-active comonomer can also be used as a functional Raman tag, but will show cell background.

Particles incorporating Raman tags can be imaged in a cellular environment without a background by stimulated Raman scattering. The Raman-tag does not degrade or leach from the particle, and stimulated Raman scattering imaging is free from photobleaching, or blinking, which can be common problems with organic fluorophores or quantum dots, respectively.

The particles can be conjugated at their periphery with a wide range of biomolecules, including nucleic acids, primarily by electrostatic interactions, and antibodies through surface active groups (e.g., either covalent or bioaffinity interactions). These particles can be functionalized with an N-hydroxysuccinimide esters, or any other suitable chemical linkers such as azides, alkynes, carboxylic acids or biotin, at their periphery permitting conjugation with antibodies bearing primary amine groups.

Once conjugated with an antibody the particles will be targeted to specific cell membrane receptors. These particles can serve as cancer marker imaging agents and image contrast agents. The particles do not display any cytotoxicity. Incorporation of multiple Raman tags across different particles can enable a multiplexing strategy, facilitating the detection of many different cells or cellular components in a single experiment. These exemplary functional nanoparticles can be useful in cellular mechanistic studies, as gene therapy delivery agents, or in diagnostic applications.

To address challenges in current systems, a polymerizable monomer can be utilized based on the tris(dialkylamino) cyclopropenium (“CP”) ion. (See, e.g., Reference 12). The aromatic CP ion can be stabilized by resonance charge delocalization through the amino substituents, yielding electron-rich stable cations. (See, e.g., References 13 and 14). CP ions have previously found applications as ionic liquids (see, e.g., Reference 15), organocatalysts (see, e.g., Reference 16), and transition-metal ligands. (See, e.g., Reference 17).

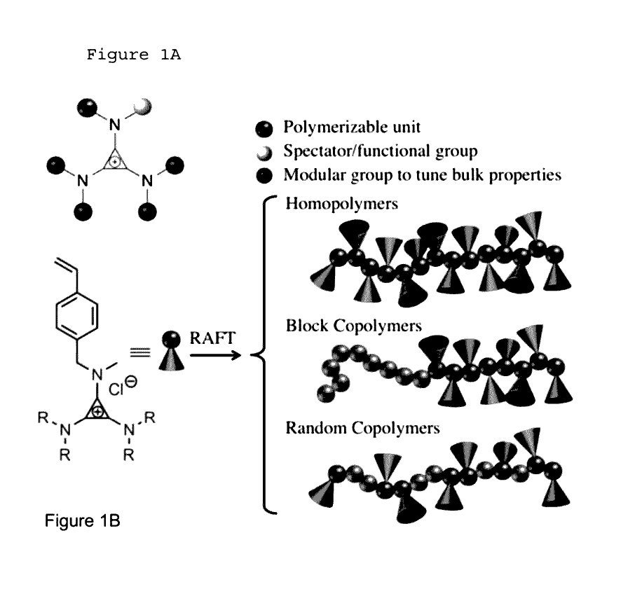

In contrast to other cationic polyelectrolytes, the cyclopropenim (“CP”) ion can have a carbon-centered formal charge (see, e.g., Reference 18) and can offer multiple handles through which to tune the physical and electronic properties. (See, e.g., Reference 12) (See e.g., an exemplary diagram shown FIG. 1A). Using the exemplary cationic monomer in reversible-deactivation radical polymerization (“RDRP”), a new exemplary polyelectrolyte family of homopolymers, statistical copolymers and diblock copolymers was generated. (See, e.g., an exemplary diagram shown FIG. 1B (which shows reversible addition—fragmentation chain transfer (“RAFT”) polymerization). The Raman-active nanoparticles that are formed are free of heavy metals.

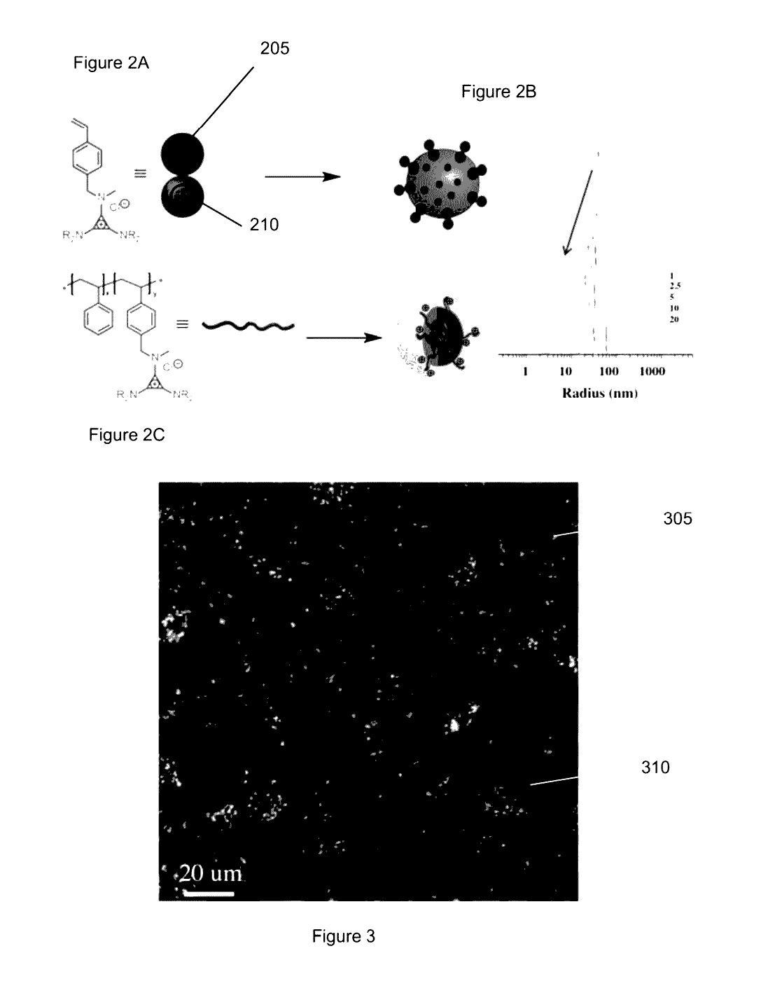

As shown in the exemplary diagram of FIG. 2A, CP-based materials, either monomers or block copolyelectrolytes, can be used in a modular surfactant-free synthesis of surface-charged nanoparticles. As CP monomers contain hydrophobic (e.g., element 205) and hydrophilic (e.g., element 210) regions, they self-assemble at oil-in-water interfaces functioning both as emulsion stabilizers and polymerizable monomers. (See e.g., Reference 25). The hydrodynamic diameters of the nanoparticles can be reliably tuned from 30-100 nm simply by varying the feed ratio of CP monomer. (See, e.g., an exemplary diagram shown in FIG. 2B (hydrophilic CP ion on the outer surface of the nanoparticle, while the hydrophobic styrene chain polymer is on the interior of the nanoparticle)). Achieving this domain size can be beneficial as it can be used for in vitro and in vivo applications. (See, e.g., Reference 26 and 27). Traditional methods that attempt to achieve nanoparticles in the sub-100 nm range utilize surfactants which can leach from particles. (See, e.g., Reference 28).

As CP can be a remarkably stable carbocation (a molecule in which a carbon atom bears three bonds and a positive charge), the exemplary nanoparticles can retain their charge and resist flocculation over a wide pH range. The modular synthesis can facilitate introduction of a variety of R-groups onto the CP monomer varying solubility and electrostatic interactions. Indeed, particle diameter and surface charge were found to correlate with R-group structure. Substituted R-groups can include hydrophobic (e.g. isopropyl, Cyclohexyl, piperidinyl) as well as hydrophilic (e.g. morpholinyl).

In addition to monomers, e.g., block copolymer electrolytes (BCPEs) can be used in the nanoparticle synthesis, facilitating a direct comparison of monomer- and BCPE-derived nanoparticle formulations. Particles derived from BCPEs possess a distinct corona, with charged moieties extending into solution. (See e.g., an exemplary diagram shown FIG. 2C (which shows the positively-charged cyclopropenium ion extended to create a corona surrounding the nanoparticles because of the additional block copolymer that has been introduced between the CP ion and the hydrophobic styrene chain polymer). As the amount of surfactant, either CP monomer or BCPE, added increases the size of the particle has been demonstrated to decrease. This access to unique particle geometries based on the nature of the emulsion stabilizer reinforces the modularity of the exemplary system. (See e.g., Reference 25).

The modular synthesis of stable cationically-charged particles can facilitate applications, such as gene-delivery vectors (see, e.g., Reference 28) and image contrast agents. (See, e.g., Reference 5). Executing the emulsion polymerization with a Raman-active comonomer can yield stable cationic nanoparticles visible by Raman spectroscopy (emulsion polymerization can be carried out according to the protocol described in International Patent Application, PCT/US2015/044331, e.g., with the introduction of a Raman-active comonomer to that protocol). The styrene comonomer can be replaced by a styrenic derivative bearing a Raman tag (e.g., either alkyne, nitrile, or Carbon-deuterium bond). Replacing as little as about 25% of the styrene comonomer with a Raman-active styrenic-derivative can yield particles with high intensity Raman signatures.

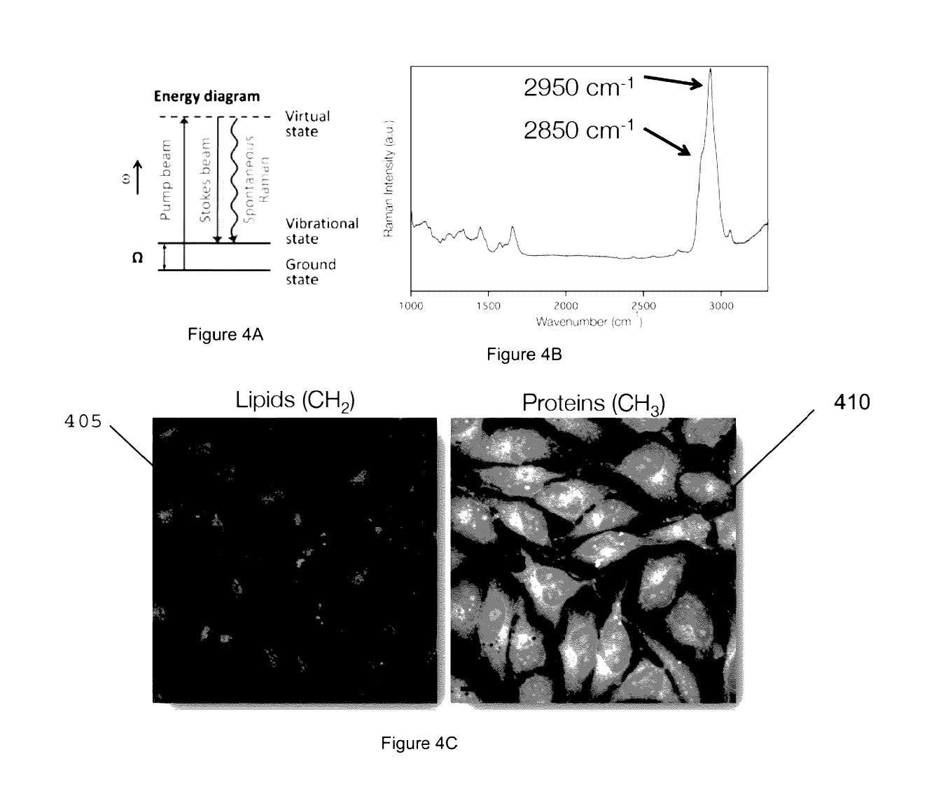

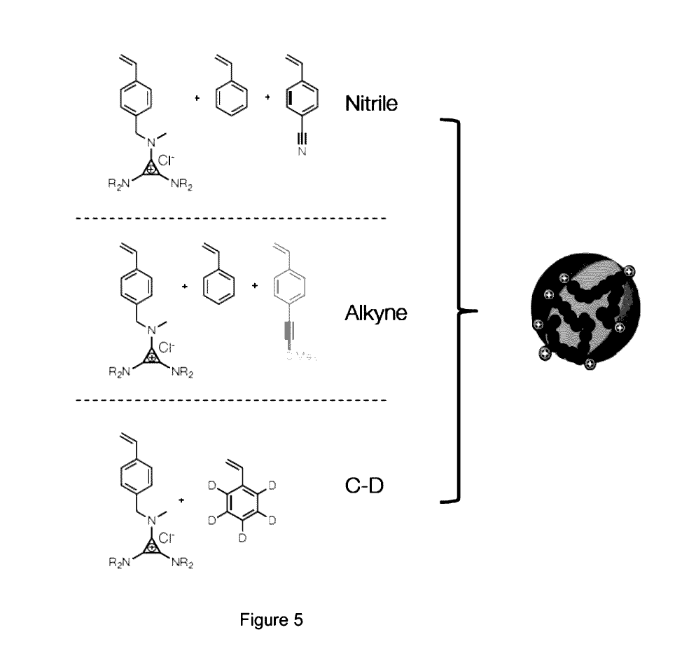

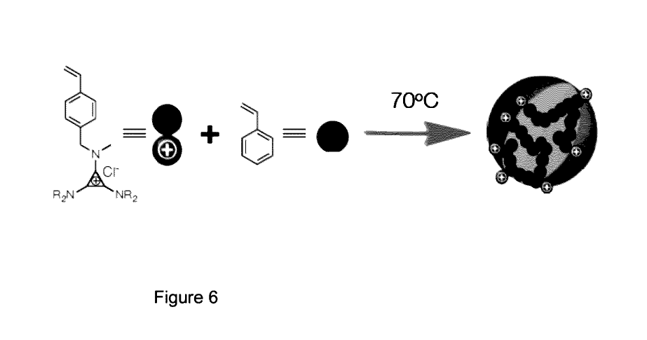

In the exemplary case of the carbon-deuterium tag, all of the styrene comonomer can be replaced by deuterated styrene (e.g., either d5 or d8) and then this monomer can be employed in the synthesis. The modular synthesis of the nanoparticles can proceed in the same fashion as syntheses without Raman-active comonomers. (See e.g., image shown in FIG. 3). For example, FIG. 3 shows an image illustrating CP nanoparticles 305 and lipids 310 according to an exemplary embodiment of the present disclosure. Exploiting the stimulated Raman scattering procedure (see e.g., Reference 29), label-free imaging of functional nanoparticles can be achieved. Multiple Raman-active nanoparticles can be developed and utilized in vivo for targeted imaging with antibody coupling. FIG. 4A shows the energy diagram for stimulated Raman scattering, where two laser beams, the stokes and pump beam, coincide on the sample. FIG. 4B shows a graph of the spontaneous Raman spectra of HeLa cells. FIG. 4C show a set of images of live HeLa cells taken stimulating vibrations corresponding to methylene vibrations (e.g., element 405) and methyl vibrations (e.g., element 410). FIG. 5 shows a set of diagrams of three different monomer compositions all of which, when combined together, can give rise to a stable positively-charged nanoparticle. FIG. 6 shows a diagrams of two exemplary monomers, and an image representation of each, that are mixed together and heated at 70° C. to yield unlabeled nanoparticles.

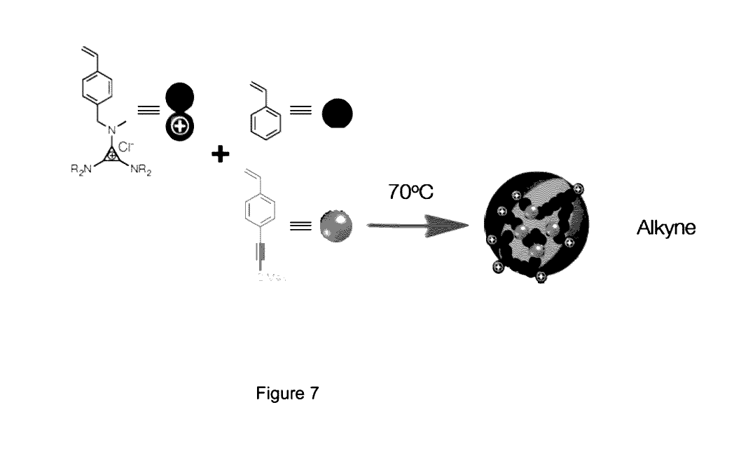

FIG. 7 shows an exemplary diagram illustrating the synthesis of an exemplary nanoparticle with an alkyne tag according to an exemplary embodiment of the present disclosure. This exemplary synthesis can be accomplished by combining 5 weight % (wt %) of the cyclopropenium monomer with 70 wt % styrene and 25 wt % trimethyl((4-vinylphenyl)ethynyl)silane and a radical initiator (e.g. V-50) for 6-24 hours at 70° C.

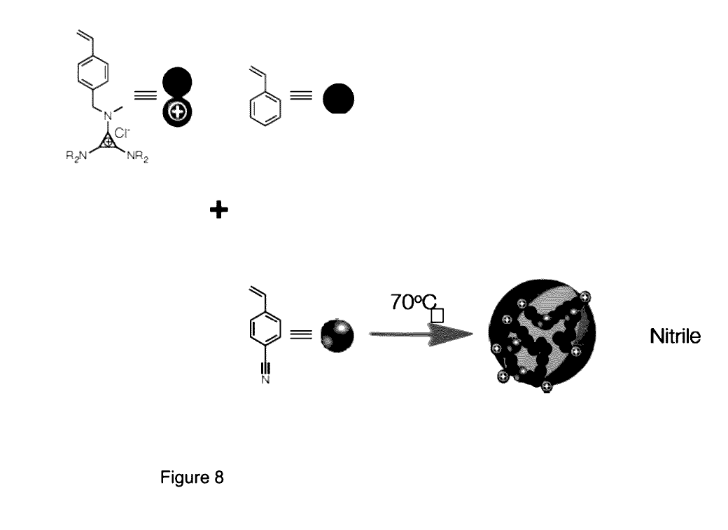

FIG. 8 shows an exemplary diagram illustrating the synthesis of a nanoparticle with a nitrile tag according to an exemplary embodiment of the present disclosure. This exemplary synthesis can be accomplished by combining 5 wt % of the cyclopropenium monomer with 70 wt % styrene and 25 wt % cyanostyrene and a radical initiator (e.g. V-50) for 6-24 hours at 70° C.

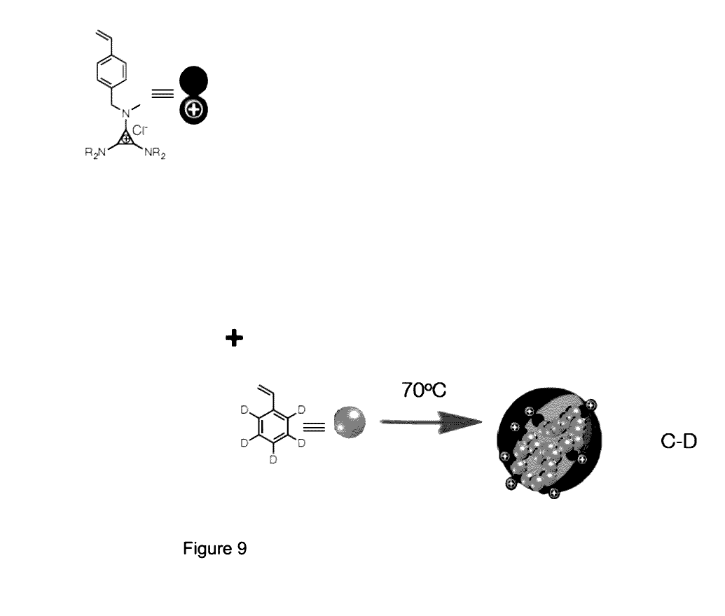

FIG. 9 shows an exemplary diagram illustrating the synthesis of a nanoparticle with a carbon-deuterium tag according to an exemplary embodiment of the present disclosure. This exemplary synthesis can be accomplished by combining 5 wt % of the cyclopropenium monomer with 95 wt % d₅-styrene and a radical initiator (e.g. V-50) for 6-24 hours at 70° C.

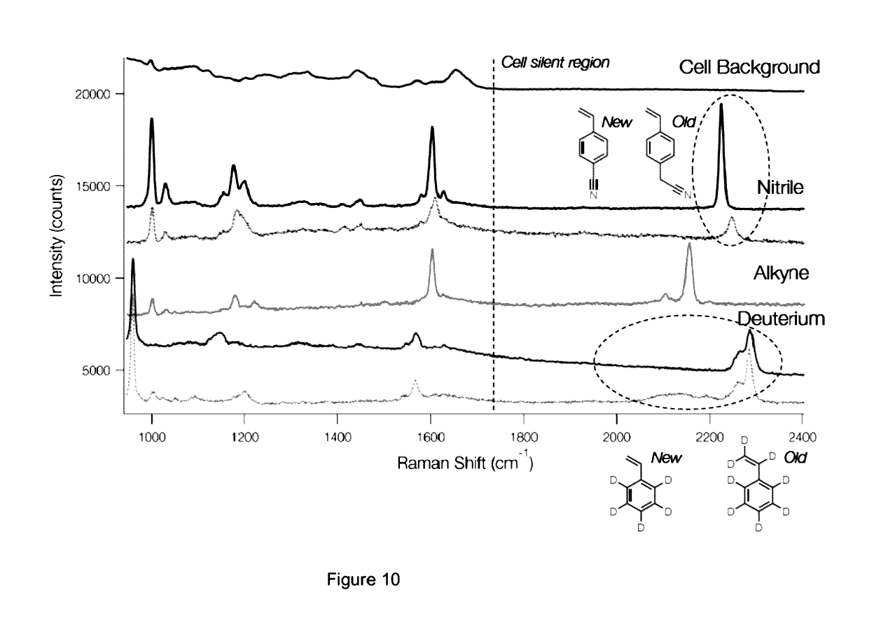

FIG. 10 shows an exemplary graph illustrating the Raman spectra for all synthesized nanoparticles according to an exemplary embodiment of the present disclosure. For example, the cell silent region is marked as greater than or equal to 1750 cm⁻¹, this is the region of cells where no Raman vibrations are naturally present. FIG. 10 also illustrates the contrasts of the Raman vibrations of different Raman-active comonomers that can be incorporated into the nanoparticle reaction. “New” and “old” are two different monomers that can be used in the synthesis of a nanoparticle with that specific Raman tag. Monomers marked “new” give higher Raman intensity and better resolution from other signals.

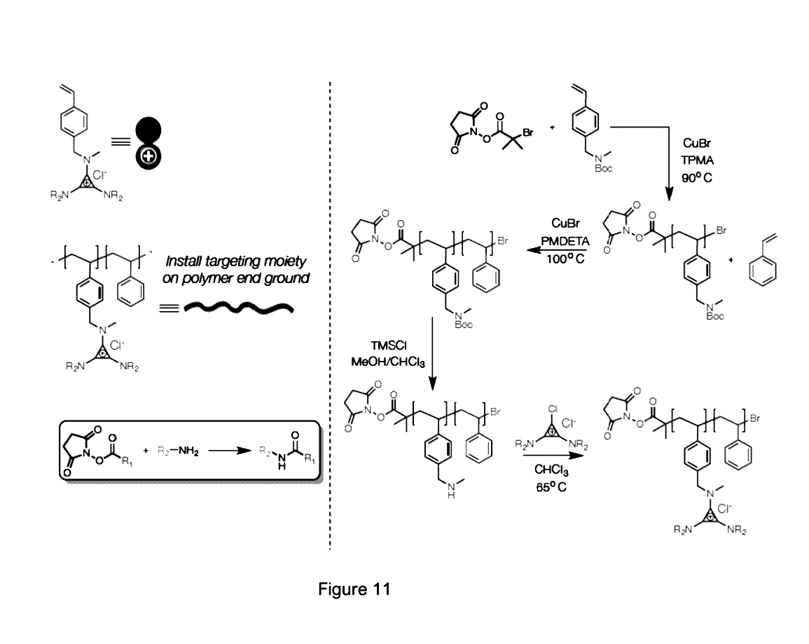

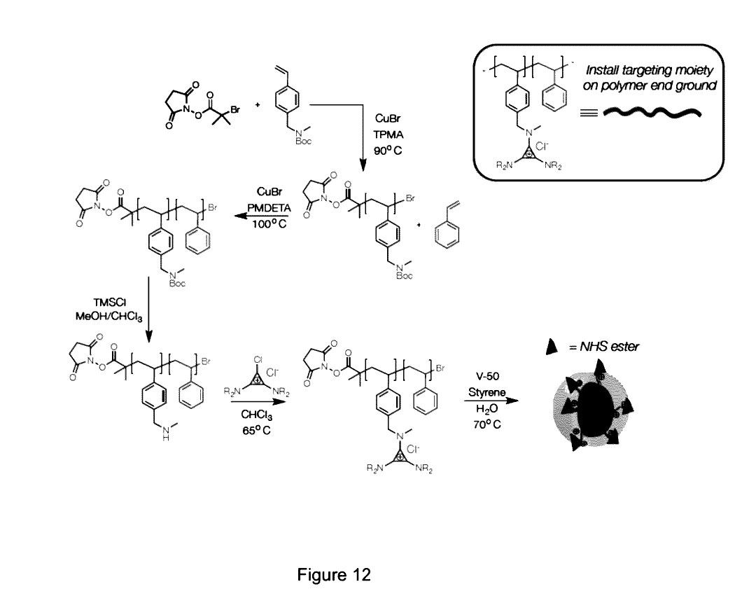

FIG. 11 shows an exemplary diagram illustrating a synthetic procedure for designing a block copolymer that has an N-hydroxysuccinimide ester tag by starting with a N-hydroxysuccinimide atom-transfer radical polymerization initiator and polymerizing two monomers in sequence according to an exemplary embodiment of the present disclosure. For example, the first polymerization proceeds with Copper (I) bromide, tris(2-pyridylmethyl)amine (“TPMA”) at 90° C. The second polymerization proceeds with Copper (I) bromide, N, N,N′,N″,N″-pentamethyldiethylenetriamine (“PMDETA”) at 100° C.

FIG. 12 shows an exemplary diagram illustrating the synthetic scheme procedure from FIG. 11 with an additional step of synthesizing the nanoparticle that has the N-hydroxysuccinimide ester tags on the periphery according to an exemplary embodiment of the present disclosure.

### Exemplary Synthesis of Nanoparticles

Particles were synthesized using the following exemplary general procedure that was scaled accordingly using about 1-20 wt % monomer (e.g., relative to styrene), styrene, 2,2-azobis(2-methylpropionamidine) dihydrochloride (e.g., V-50), and water. The final solution was scaled to about 10 g, with about a 10% monomer content. The cyclopropenium cation (“CPR”) monomer was dissolved in styrene, and an initiator was dissolved separately in about 1 mL of water. The remaining volume of water was added to the monomer solution, and the V-50 solution was added to the monomer suspension. The mixture was vortexed for about 30 seconds. The solution was added to a two-neck flask fitted with a condenser and stir bar and was sparged with N2 or Argon for about 10 minutes. The solution was stirred at about 70° C. for about 6-16 hours.

### Exemplary Synthesis of Alkyne-Tag Nanoparticles

Exemplary synthesis of a nanoparticle with an alkyne tag can be performed by combining about 50 mg (e.g., 5 wt %) of the cyclopropenium monomer with about 700 mg (e.g., 70 wt %) styrene and 250 mg (25 wt %) trimethyl((4-vinylphenyl)ethynyl)silane and about 5-10 mg 2,2-azobis(2-methylpropionamidine) dihydrochloride (e.g., V-50), and about 9 mL water. The mixture was vortexed for about 30 seconds. The solution was then added to a two-neck flask fitted with a condenser and stir bar and was sparged with Argon for about 10 min. The solution was stirred at about 70° C. for about 6-24 hours.

### Exemplary Synthesis of Nitrile-Tag Nanoparticles

Exemplary synthesis of a nanoparticle with an nitrile tag can be performed by combining 50 mg (e.g., 5 wt %) of the cyclopropenium monomer with about 700 mg (e.g., 70 wt %) styrene and about 250 mg (e.g., 25 wt %) cyanostyrene and about 5-10 mg 2,2-azobis(2-methylpropionamidine) dihydrochloride (e.g., V-50), and about 9 mL water. The mixture was vortexed for about 30 seconds. The solution was then added to a two-neck flask fitted with a condenser and stir bar and was sparged with Argon for about 10 min. The solution was stirred at about 70° C. for about 6-24 hours.

### Exemplary Synthesis of Deuterated Nanoparticles

Synthesis of a nanoparticle with a carbon-deuterium tag can be performed by combining about 50 mg (e.g., 5 wt %) of the cyclopropenium monomer with about 950 mg (e.g., 95 wt %) deuterated styrene and about 5-10 mg 2,2-azobis(2-methylpropionamidine) dihydrochloride (e.g., V-50), and about 9 mL water. The mixture was vortexed for about 30 seconds. The solution was then added to a two-neck flask fitted with a condenser and stir bar and was sparged with Argon for about 10 min. The solution was stirred at about 70° C. for about 6-24 hours.

### Exemplary Functionalization of Raman-Active Nanoparticles as Theranostic Agents

Nanoparticles functionalized with nucleic acids, drugs, or antibodies can be incubated in vitro in cells to assess activity, including transfection efficacy, drug delivery, or specific targeting. Combined with the Raman imaging of the nanoparticles, these exemplary systems can serve as theranostic agents. For example, the most prevalent human-derived immortal cell lines which can be tested include HeLa (cervical cancer), 293T (embryonic kidney), HT1080 (fibrous sarcoma) and DU145 (prostate cancer).

Exemplary Conjugation of Raman Active Nanoparticles with Nucleic Acids

The nanoparticles can be conjugated at their periphery with nucleic acids (e.g., DNA, plasmid DNA, mRNA, siRNA, miRNA or similar) through electrostatic interactions. This exemplary conjugation can be measured by dynamic light scattering (“DLS”) and transmission electron microscopy (“TEM”) for nanoparticle size modulation, and by gel retardation experiments where post-conjugation all nucleic acid can be bound by the nanoparticle and may no longer be free to migrate down a cell.

In some exemplary embodiments of the present disclosure, the therapeutic RNAs can be siRNAs. In some exemplary embodiments of the present disclosure, the therapeutic RNAs can be precursor miRNAs. In some exemplary embodiments of the present disclosure, the therapeutic RNAs can be mature single-stranded miRNAs. In some exemplary embodiments of the present disclosure, the therapeutic RNAs can be mature double-stranded miRNAs. In some exemplary embodiments of the present disclosure, the therapeutic RNAs can be antisense RNAs.

A non-limiting list of compositions and/or procedures that may be used to inhibit gene expression or biological activity can include: (i) antisense oligonucleotides, (ii) ribozymes, (iii) RNA interference, (iv) CRISPR modification of the genomic nucleotide sequence in cancer tissue and (v) small molecule drugs or antibodies against one or more proteins expressed a biomarkers by tumors or other cellular targets (e.g. inflammatory disease cells, necrotic tissue, etc.). Such compositions for inhibition of gene expression or biological activity can be delivered by procedures known to one of ordinary skill in the art, such as viral vectors.

As used herein, the phrase “pharmaceutically acceptable carrier” can include any and all molecular entities and compositions that can be of sufficient purity and quality for use in the formulation of a composition or medicament of the present disclosure and that, when appropriately administered to an animal or a human, may not produce an adverse, allergic or other untoward reaction. Since both human use (e.g., clinical and over-the-counter) and veterinary use can be equally included within the scope of the present disclosure, a pharmaceutically acceptable formulation can include a composition or medicament for either human or veterinary use. In one exemplary embodiment of the present disclosure, the pharmaceutically acceptable carrier can be water or a water based solution. In another exemplary embodiment of the present disclosure, the pharmaceutically acceptable carrier can be a non-aqueous polar liquid such as dimethyl sulfoxide, polyethylene glycol and polar silicone liquids. In another exemplary embodiment of the present disclosure, the carrier can be liposomal or polymeric agents. The use of such media and agents with pharmaceutically active substances is well known in the art. Except insofar as any conventional media or agent can be incompatible with the Raman-active nanoparticle-nucleic acid conjugates, its use in the therapeutic compositions is contemplated.

The pharmaceutical composition, according to some exemplary embodiments of the present disclosure, can be formulated in a dosage form for the desired route of administration. The amount of the Raman-active nanoparticle-nucleic acid conjugates, which can be combined with the carrier material to produce a single dosage form, can vary depending upon the host being treated, and the particular mode of administration. The amount of the Raman-active nanoparticle-nucleic acid conjugates that can be combined with the carrier material to produce a single dosage form can generally be that amount of the conjugate which produces a therapeutic effect.

Formulations suitable for parenteral administration can include, for example, the Raman-active nanoparticle-nucleic acid conjugates in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which can contain sugars, alcohols, antioxidants, buffers, bacteriostats, solutes which can render the formulation isotonic with the blood of the intended recipient or suspending or thickening agents. Examples of suitable aqueous and nonaqueous carriers, which can be employed in the pharmaceutical compositions, can include water, ethanol, polyols (e.g., glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. The pharmaceutical compositions can also contain adjuvants such as preservatives, wetting agents, emulsifying agents and dispersing agents. Prevention of the action of microorganisms upon the subject compositions can be ensured by the inclusion of various antibacterial and antifungal agents, for example, paraben, chlorobutanol, phenol sorbic acid and the like. It can also be desirable to include isotonic agents, such as sugars, sodium chloride and the like into the compositions.

Various delivery systems are known and can be used to administer a therapeutic agent useful for preventing or treating cancer, for example, encapsulation in liposomes, microparticles, microcapsules, recombinant cells capable of expressing the composition, receptor-mediated endocytosis, construction of a nucleic acid as part of a retroviral or other vector, etc. The pharmaceutical composition can be administered intravenously, intra-arterially or in other suitable ways to a subject in need of such treatment. Administration of the pharmaceutical composition can occur for a period of seconds, hours, days or weeks depending on the purpose of the pharmaceutical composition usage. In some exemplary embodiment of the present disclosure, the pharmaceutical composition of the present application can be administered intravenously.

In some exemplary embodiment of the present disclosure, administration of the pharmaceutical composition can be performed parenterally. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The dosage level of the pharmaceutical composition of the present disclosure can depend upon a variety of factors including the activity of the particular composition of the present invention employed, the route of administration, the time of administration, the rate of excretion or metabolism of the particular composition being employed, the duration of the treatment, other drugs, compounds and/or materials used in combination with the particular composition employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compositions of the invention employed in the pharmaceutical composition at levels lower than that required to achieve the desired therapeutic effect and then gradually increasing the dosage until the desired effect is achieved. As a general proposition, the therapeutically effective amount of Raman-active nanoparticle-nucleic acid conjugate can be administered in the range of about 0.1 μg/kg body weight/day to about 100000 mg/kg body weight/day whether by one or more administrations. In some embodiments, the range of each active agent administered daily can be from about 100 μg/kg body weight/day to about 50 mg/kg body weight/day, 100 μg/kg body weight/day to about 10 mg/kg body weight/day, 100 μg/kg body weight/day to about 1 mg/kg body weight/day, 100 μg/kg body weight/day to about 10 mg/kg body weight/day, 500 μg/kg body weight/day to about 100 mg/kg body weight/day, 500 μg/kg body weight/day to about 50 mg/kg body weight/day, 500 μg/kg body weight/day to about 5 mg/kg body weight/day, 1 mg/kg body weight/day to about 100 mg/kg body weight/day, 1 mg/kg body weight/day to about 50 mg/kg body weight/day, 1 mg/kg body weight/day to about 10 mg/kg body weight/day, 5 mg/kg body weight/dose to about 100 mg/kg body weight/day, 5 mg/kg body weight/dose to about 50 mg/kg body weight/day, 10 mg/kg body weight/day to about 100 mg/kg body weight/day, and 10 mg/kg body weight/day to about 50 mg/kg body weight/day. In some embodiments, the Raman-active nanoparticle-nucleic acid conjugate can be administrated daily at the above-described doses for 1, 2, 3, 4, 5, 6, 7, 8, 9, 10, 11, 12, 13 or 14 days. In some embodiments, Raman-active nanoparticle-nucleic acid conjugate can be administrated daily at the above-described doses for a period prescribed by the physician or veterinarian to chronically treat a medically necessary condition. One of ordinary skill will understand that the choice of dosage level or form of administration is not limiting on the present disclosure.

Exemplary Conjugation of Raman Active Nanoparticles with Drug or Antibodies

The nanoparticle periphery can also be conjugated with a drug or antibody via covalent coupling. Functionalizing the surface of nanoparticles with reactive moieties, such as N-hydroxysuccinimide esters, biotin, or azides, can facilitate coupling with molecules bearing partner reactivity (e.g., amines, streptavidin and alkynes, respectively). In addition, a drug can be conjugated to nanoparticle surface via a pH sensitive linker that can hydrolyze in acidic conditions. Thus, the drug can be cleaved and released from the nanoparticle in the acidic intracellular environment of the endosome. The functionalizations can be assessed by DLS or TEM for size changes, after purification by dialysis to remove uncoupled moieties. For example, an antibody, Ep-CAM, can be used to specifically functionalize the surface of breast cancer lines (e.g., MCF-7). An exemplary can illustrate functionalized nanoparticles, specifically labeling cancer lines bearing the necessary surface structures. As these exemplary nanoparticles can be robustly imaged, this exemplary methodology can facilitate screening protocols.

As used herein, the term “cancer” or “tumor” refers to any of the various or pre-malignant (e.g., benign tumor and atypical hyperplasia) or malignant (e.g., cancer) neoplasms characterized by the proliferation of cells that have the capability to invade surrounding tissue and/or metastasize to new colonization sites, including but not limited to leukemias, lymphomas, carcinomas, melanomas, sarcomas, germ cell tumors and blastomas. Exemplary cancers include cancers of the brain, bladder, breast, cervix, colon, head and neck, kidney, lung, non-small cell lung, mesothelioma, ovary, prostate, stomach and uterus, leukemia and medulloblastoma. Cancer can originate from any cell type or tissue found in a mammal, including, but not limited to hepatic, skin, breast, prostate, neural, optic, intestinal, cardiac, vasculature, lymph, spleen, renal, bladder, lung, muscle, connective, tissue, pancreatic, pituitary, endocrine, reproductive organs, bone, and blood.

As used herein, the terms “subject,” “individual,” and “animal” are used interchangeably herein to refer to a vertebrate, preferably a mammal. The term “mammal” or “mammalian” includes, but is not limited to, murines, simians, humans, farm animals, sport animals and pets. Tissues, cells and their progeny of a biological entity obtained in vivo or cultured in vitro can also be encompassed.

As used herein, the terms “treat,” “treating” and “treatment” refer to the eradication, reduction or amelioration of symptoms of a disease or disorder, particularly, the eradication, removal, modification, or control of primary, regional, or metastatic cancer tissue that results from the administration of one or more therapeutic agents. In certain embodiments, such terms refer to the minimizing or delaying the spread of cancer resulting from the administration of one or more therapeutic agents to a subject with such a disease.

As used herein, the term “therapeutic agent” refers to any agent that can be used in the prevention, treatment, or management of a disease or disorder associated with cancer. As used herein, a “therapeutically effective amount” refers to that amount of the composition sufficient to treat or manage a disease or disorder associated with cancer and, preferably, the amount sufficient to destroy, modify, control or remove primary, regional or metastatic cancer tissue. The appropriate dosage (“therapeutically effective amount”) of the composition will depend, for example, on the severity and course of the cancer being treated, the mode of administration, the bioavailability of the therapeutic agent(s), previous therap(ies), the age and weight of the patient, the patient's clinical history and response to the composition, the type of composition used, discretion of the attending physician, etc. The composition is suitably administered to the patent at one time or over a series of treatments and may be administered to the patient at any time from diagnosis onwards. The composition may be administered as the sole treatment or in combination with other drugs or therapies useful in treating cancer. When used with other drugs, the composition may be used at a lower dose to reduce toxicities and/or side effects.

One of ordinary skill will also understand that the dosage and other formulatory requirements for antibody-drug conjugates may be varied as discussed herein with respect to Raman-active-nanoparticle-nucleic acid conjugates and vice versa. Anticancer compositions may be administered orally or parenterally. Anti-cancer agents may be formulated as tablets, capsules, granules, powders, sublingual tablets, suppositories, ointments, injections, emulsions solutions, formulated as suspensions, syrups, sprays, etc. The compositions can be prepared by known techniques using a carrier in a conventional pharmaceutically acceptable used for each formulation. Examples of pharmaceutically acceptable carriers can be excipients, binders, disintegrating agents, lubricants, preservatives, antioxidants, isotonic agents, a buffer, a sweetener, a solubilizer, a base, a dispersing agent, a wetting agent, and a suspending agent, stabilizer, coloring agent, etc.

The dosage of the specific type of mammal including humans, to be treated in the anticancer composition can be varied according to weight, sex, severity of disease, depending on the judgment of the doctor. The total daily dose may be administered depending on the degree of the disease, judgment of the physician at a time or by dividing circuit. As a general proposition, the therapeutically effective amount(s) of the above described compositions will be in the range of about 1 ng/kg body weight/day to about 100 mg/kg body weight/day whether by one or more administrations. In a particular embodiments, each therapeutic agent is administered in the range of from about 1 ng/kg body weight/day to about 10 mg/kg body weight/day, about 1 ng/kg body weight/day to about 1 mg/kg body weight/day, about 1 ng/kg body weight/day to about 100 μg/kg body weight/day, about 1 ng/kg body weight/day to about 10 μg/kg body weight/day, about 1 ng/kg body weight/day to about 1 μg/kg body weight/day, about 1 ng/kg body weight/day to about 100 ng/kg body weight/day, about 1 ng/kg body weight/day to about 10 ng/kg body weight/day, about 10 ng/kg body weight/day to about 100 mg/kg body weight/day, about 10 ng/kg body weight/day to about 10 mg/kg body weight/day, about 10 ng/kg body weight/day to about 1 mg/kg body weight/day, about 10 ng/kg body weight/day to about 100 μg/kg body weight/day, about 10 ng/kg body weight/day to about 10 μg/kg body weight/day, about 10 ng/kg body weight/day to about 1 μg/kg body weight/day, 10 ng/kg body weight/day to about 100 ng/kg body weight/day, about 100 ng/kg body weight/day to about 100 mg/kg body weight/day, about 100 ng/kg body weight/day to about 10 mg/kg body weight/day, about 100 ng/kg body weight/day to about 1 mg/kg body weight/day, about 100 ng/kg body weight/day to about 100 μg/kg body weight/day, about 100 ng/kg body weight/day to about 10 μg/kg body weight/day, about 100 ng/kg body weight/day to about 1 μg/kg body weight/day, about 1 μg/kg body weight/day to about 100 mg/kg body weight/day, about 1 μg/kg body weight/day to about 10 mg/kg body weight/day, about 1 μg/kg body weight/day to about 1 mg/kg body weight/day, about 1 μg/kg body weight/day to about 100 μg/kg body weight/day, about 1 μg/kg body weight/day to about 10 μg/kg body weight/day, about 10 μg/kg body weight/day to about 100 mg/kg body weight/day, about 10 μg/kg body weight/day to about 10 mg/kg body weight/day, about 10 μg/kg body weight/day to about 1 mg/kg body weight/day, about 10 μg/kg body weight/day to about 100 μg/kg body weight/day, about 100 μg/kg body weight/day to about 100 mg/kg body weight/day, about 100 μg/kg body weight/day to about 10 mg/kg body weight/day, about 100 μg/kg body weight/day to about 1 mg/kg body weight/day, about 1 mg/kg body weight/day to about 100 mg/kg body weight/day, about 1 mg/kg body weight/day to about 10 mg/kg body weight/day, about 10 mg/kg body weight/day to about 100 mg/kg body weight/day.

In other particular exemplary embodiments of the present disclosure, the compositions can be administered at a dose of about 0.0006 mg/day, 0.001 mg/day, 0.003 mg/day, 0.006 mg/day, 0.01 mg/day, 0.03 mg/day, 0.06 mg/day, 0.1 mg/day, 0.3 mg/day, 0.6 mg/day, 1 mg/day, 3 mg/day, 6 mg/day, 10 mg/day, 30 mg/day, 60 mg/day, 100 mg/day, 300 mg/day, 600 mg/day, 1000 mg/day, 2000 mg/day, 5000 mg/day or 10,000 mg/day. As expected, the dosage(s) will be dependent on the condition, size, age and condition of the patient.

Linkers for conjugation may be cleavable or non-cleavable. General linkers may be amide, thiourea, haloacetyl-thioether, maleimide-thioether, hydrazone, disulfide, Val-Cit dipeptide, copper-free click ((Bertozzi (2007) PNAS, 104(43), 16793), traceless linkers (Nature Protocols, 2013, 8, 2079), etc. In particular, where an antibody or a drug is conjugated with Raman-active nanoparticles the linker may be a bifunctional linker that links both a drug and an antibody to the Raman-active nanoparticle. Spacers may also be used as required. One of ordinary skill will understand that the choice of linker for conjugation is not limiting on the invention.

Anticancer cytotosic agents which may be conjugated with the Raman-active nanoparticles include anthracyclines (e.g. doxorubicin), vinca-alkaloids (e.g. vinblastine), taxanes (e.g. paclitaxel, docetaxel), platinum derivatives (e.g. cisplatin), pyrimidine analogues (e.g. gemcitabine), purine analogues (e.g. 6-mercaptopurine), alkylating agents (e.g. cyclophosphamide) and others, such as actinomycin D or tamoxifen. One of ordinary skill will understand that the choice of anticancer cytotoxic agent is not limiting on the invention.

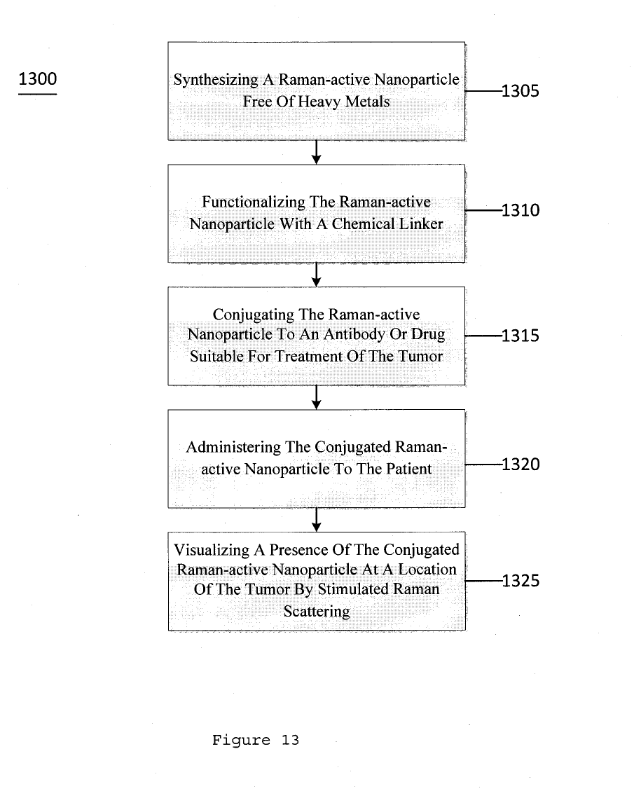

FIG. 13 shows an exemplary flow diagram of an exemplary method 1300 for treating and visualizing a tumor in a patient in need thereof. For example, at procedure 1305, a Raman-active nanoparticle free of heavy metals can be synthesized, where the Raman-active nanoparticle can include a hydrophilic cyclopropenium cation and one or more copolymers formed from a hydrophobic organic polymerizable molecule, and where the hydrophobic organic polymerizable molecule can be one of a styrenic derivative or a methacrylate derivative. At procedure 1310, the Raman-active nanoparticle can be functionalized with a chemical linker. At procedure 1315, the Raman-active nanoparticle can be conjugated to an antibody or drug suitable for treatment of the tumor. At procedure 1320, the conjugated Raman-active nanoparticle can be administered to the patient. At procedure 1325, a presence of the conjugated Raman-active nanoparticle can be visualized at a location of the tumor by stimulated Raman scattering.

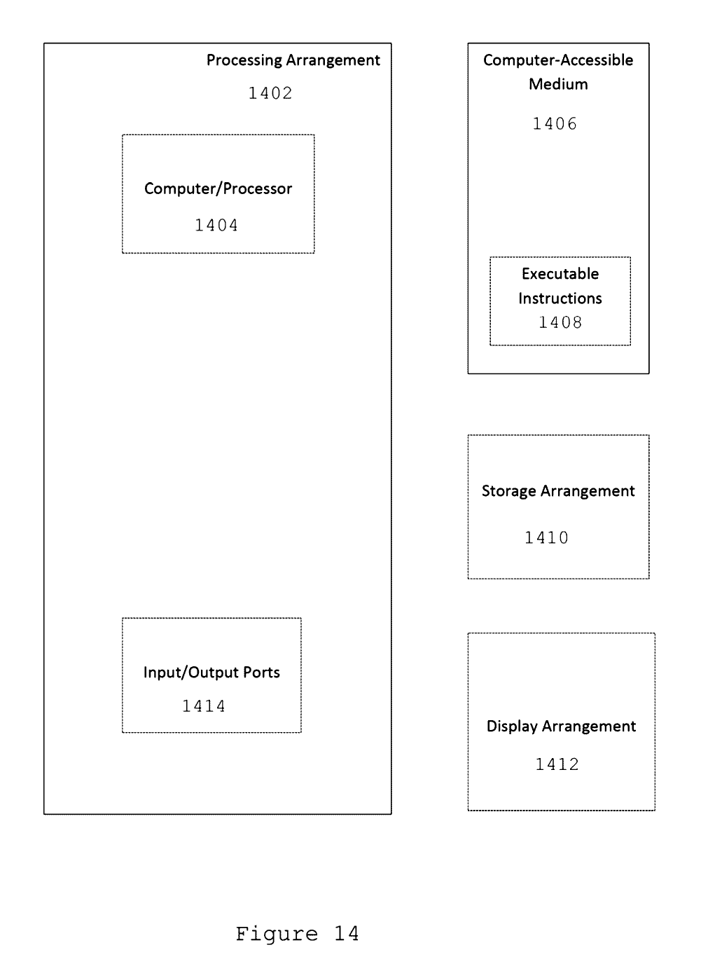

FIG. 14 shows a block diagram of an exemplary embodiment of a system according to the present disclosure. For example, exemplary procedures in accordance with the present disclosure described herein can be performed by a processing arrangement and/or a computing arrangement 1402. Such processing/computing arrangement 1402 can be, for example entirely or a part of, or include, but not limited to, a computer/processor 1404 that can include, for example one or more microprocessors, and use instructions stored on a computer-accessible medium (e.g., RAM, ROM, hard drive, or other storage device).

As shown in FIG. 14, for example a computer-accessible medium 1406 (e.g., as described herein above, a storage device such as a hard disk, floppy disk, memory stick, CD-ROM, RAM, ROM, etc., or a collection thereof) can be provided (e.g., in communication with the processing arrangement 1402). The computer-accessible medium 1406 can contain executable instructions 1408 thereon. In addition or alternatively, a storage arrangement 1410 can be provided separately from the computer-accessible medium 1406, which can provide the instructions to the processing arrangement 1402 so as to configure the processing arrangement to execute certain exemplary procedures, processes and methods, as described herein above, for example.

Further, the exemplary processing arrangement 1402 can be provided with or include an input/output arrangement 1414, which can include, for example a wired network, a wireless network, the internet, an intranet, a data collection probe, a sensor, etc. As shown in FIG. 14, the exemplary processing arrangement 1402 can be in communication with an exemplary display arrangement 1412, which, according to certain exemplary embodiments of the present disclosure, can be a touch-screen configured for inputting information to the processing arrangement in addition to outputting information from the processing arrangement, for example. Further, the exemplary display 1412 and/or a storage arrangement 1410 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference in their entireties.
1. Hawker, C. J.; Wooley, K. L. Science 2005, 309 (5738), 1200-1205.
2. Hallinan, D. T.; Balsara, N. P. Annu. Rev. Mater. Res. 2013, 43 (1), 503-525.
3. Samal, S. K.; Dash, M.; Van Vlierberghe, S.; Kaplan, D. L.; Chiellini, E.; van Blitterswijk, C.; Moroni, L.; Dubruel, P. Chem. Soc. Rev. 2012, 41 (21), 7147-7194.
4. Yuan, J.; Mecerreyes, D.; Antonietti, M. Prog. Polym. Sci. 2013, 38 (7), 1009-1036.
5. Mintzer, M. A.; Simanek, E. E. Chem. Rev. 2009, 109 (979), 259-302. (6)Qiu, B.; Lin, B.; Qiu, L.; Yan, F. J. Mater. Chem. 2012, 22, 1040.
7. Jangu, C.; Long, T. E. Polymer 2014, 55 (16), 3298-3304.
8. Ornelas-Megiatto, C.; Wich, P. R.; Fréchet, J. M. J. J. Am. Chem. Soc. 2012, 134 (4), 1902-1905.
9. Steele, B. C.; Heinzel, A. Nature 2001, 414, 345-352.
10. Sing, C. E.; Zwanikken, J. W.; Olvera, M.; Cruz, D. Nat. Mater. 2014, 13, 694-698.
11. Yin, H.; Kanasty, R. L.; Eltoukhy, A. A.; Vegas, A. J.; Dorkin, J. R.; Anderson, D. G. Nat. Rev. Genet. 2014, 15 (8), 541-555.
12. Jiang, Y.; Freyer, J. L.; Cotanda, P.; Brucks, S. D.; Killops, K. L.; Bandar, J. S.; Torsitano, C.; Balsara, N. P.; Lambert, T. H.; Campos, L. M. Nat. Commun. 2015, 6, 5950.
13. Yoshida, Z.; Tawara, Y. J. Am. Chem. Soc. 1971, 93 (1962), 2573-2574.
14. Bandar, J.; Lambert, T. Synth. 2013, 45, 2485-2498.
15. Curnow, O. J.; Macfarlane, D. R.; Walst, K. J. Chem. Commun. 2011, 47, 10248-10250.
16. Bandar, J. S.; Lambert, T. H. J. Am. Chem. Soc. 2012, 134, 5552-5555.
17. Bruns, H.; Patil, M.; Carreras, J.; Vazquez, A.; Thiel, W.; Goddard, R.; Alcarazo, M. Angew. Chem. Int. Ed. 2010, 49, 3680-3683.
18. Breslow, R. J. Am. Chem. Soc. 1957, 79 (1), 5318.
19. Bates, F. S.; Fredrickson, G. H. Phys. Today 1999, 52 (2), 32.
20. Knychala, P.; Banaszak, M.; Balsara, N. P. Macromolecules 2014, 47, 2529-2535.
21. Weber, R. L.; Ye, Y.; Schmitt, A. L.; Banik, S. M.; Elabd, Y. A.; Mahanthappa, M. K. Macromolecules 2011, 44 (14), 5727-5735.
22. Shi, Z.; Newell, B. S.; Bailey, T. S.; Gin, D. L. Polymer 2014, 55 (26), 6664-6671.
23. Such, G. K.; Yan, Y.; Johnston, A. P. R.; Gunawan, S. T.; Caruso, F. Adv. Mater. 2015, ASAP DOI: 10.1002/adma.201405084
24. Chai, J.; Buriak, J. M. ACS Nano 2008, 2 (3), 489-501.
25. Killops, K. L.; Brucks, S. D.; Rutkowski, K. L.; Freyer, J. L.; Jiang, Y.; Valdes, E. R.; Campos, L. M. Macromolecules 2015. Submitted.
26. Elsabahy, M.; Wooley, K. L. Chem. Soc. Rev. 2012, 41, 2545-2561.
27. Stuart, M. A. C.; Huck, W. T. S.; Genzer, J.; Müller, M.; Ober, C.; Stamm, M.; Sukhorukov, G. B.; Szleifer, I.; Tsukruk, V. V; Urban, M.; Winnik, F.; Zauscher, S.; Luzinov, I.; Minko, S. Nat. Mater. 2010, 9 (2), 101-113.
28. Ramos, J.; Forcada, J.; Hidalgo-Alvarez, R. Chem. Rev. 2014, 114 (1), 367-428.
29. Wei, L.; Yu, Y.; Shen, Y.; Wang, M.; Min, W. Proc. Natl. Acad. Sci. U.S. Pat. No. 2,013,110, 11226-11231.
30. Merkel, 0. M.; Kissel, T. J. Control. Release 2014, 190, 415-423.

What is claimed is:

1. A Raman-active nanoparticle comprising:
a hydrophilic cyclopropenium cation; and
one or more copolymers derived from a hydrophobic organic polymerizable molecule;
wherein the hydrophobic organic polymerizable molecule is a Raman-active molecule, and wherein the Ramain-active nanoparticle is free of heavy metals.

2. The Raman-active nanoparticle of claim 1, wherein the hydrophobic organic polymerizable molecule is a styrenic derivative.

3. The Raman-active nanoparticle of claim 2, wherein the styrenic derivative is one of an alkyne, a nitrile, or a deuterated styrene.

4. The Raman-active nanoparticle of claim 1, wherein the hydrophobic organic polymerizable molecule is a methacrylate derivative.

5. The Raman-active nanoparticle of claim 1, wherein the Raman-active nanoparticle further includes a block copolymer electrolyte.

6. The Raman-active nanoparticle of claim 5, wherein the Raman-active nanoparticle further includes a styrenic derivative.

7. The Raman-active nanoparticle of claim 6, wherein the styrenic derivative is at least one of an alkyne, a nitrile, or a deuterated styrene.

8. The Raman-active nanoparticle of claim 1, wherein the Raman-active nanoparticle further includes a functionalized chemical linker.

9. The Raman-active nanoparticle of claim 8, wherein the functionalized chemical linker is an N-hydroxysuccinimide ester.

10. The Raman-active nanoparticle of claim 8, wherein the Raman-active nanoparticle is conjugated to an antibody.

11. The Raman-active nanoparticle of claim 8, wherein the Raman-active nanoparticle is conjugated to a drug.

12. The Raman-active nanoparticle of claim 11, wherein the Raman-active nanoparticle is conjugated to a drug for breast cancer.

13. The Raman-active nanoparticle of claim 1, wherein the Raman-active nanoparticle further includes a nucleic acid conjugated by electrostatic interactions.

14. The Raman-active nanoparticle of claim 13, wherein the nucleic acid is RNA.

15. The Raman-active nanoparticle of claim 13, wherein the nucleic acid is DNA.

16. A method of treating and visualizing a tumor in a patient, comprising:
synthesizing a Raman-active nanoparticle free of heavy metals, wherein the Raman-active nanoparticle include a hydrophilic cyclopropenium cation and one or more copolymers formed from a hydrophobic organic polymerizable molecule, and wherein the hydrophobic organic polymerizable molecule is at least one of a styrenic derivative or a methacrylate derivative;
functionalizing the Raman-active nanoparticle with a chemical linker;
conjugating the Raman-active nanoparticle to an antibody or drug suitable for treatment of the tumor;
administering the conjugated Raman-active nanoparticle to the patient; and
visualizing a presence of the conjugated Raman-active nanoparticle at a location of the tumor by stimulated Raman scattering.

17. The method of claim 16, wherein the antibody targets cell surface proteins specifically expressed by the tumor.

18. The method of claim 16, wherein the drug is suitable for treatment of breast cancer, prostate cancer, colon cancer, liver cancer, skin cancer or lung cancer.

19. The method of claim 16, wherein the chemical linker includes one of N-hydroxysuccinimide ester, disulfide linkers, or non-cleavable methyl ester linkers.

20. The method of claim 16, wherein the styrenic derivatives include one of analkyne, a nitrile or a deuterated styrene.
